(19) United States
(12) Patent Application Publication
(10) Pub. No.: US 2010/0099880 A1
(43) Pub. Date: Apr. 22, 2010

(54) Alternate Process for Remifentanil Preparation

(76) Inventor: Brian Cheng, Chesterfield, MO (US)

Correspondence Address: Mallinckrodt Inc., 675 McDonnell Boulevard, HAZELWOOD, MO, 63042, US

(21) Appl. No.: 12/443,513

(22) PCT Filed: Sep. 20, 2007
(86) PCT No.: PCT/US07/20430

§ 371 Date: Mar. 30, 2009

Related U.S. Application Data
(60) Provisional application No. 60/849,639, filed on Oct. 5, 2006.

(51) Int. Cl. C07D 211/98; C07D 211/60

(52) U.S. Cl. 546/244; 546/245

(57) ABSTRACT

An alternate process for synthesizing opiate or opioid analgesics and anesthetics, and intermediates thereof is provided. In particular, a process of synthesizing synthetic opiate or opioid compounds such as, for example, remifentanil, carfentanil, sufentanil, fentanyl, and alfentanil are disclosed.

### FIELD OF THE INVENTION

The present invention generally relates to a process for synthesizing opiate or opioid analgesics and anesthetics, and precursors thereof. In particular, the present invention relates to a process for synthesizing opiate or opioid compounds such as, for example, remifentanil, carfentanil, sufentanil, fentanyl, and alfentanil. In particular, the present invention relates to an alternate process for preparation of remifentanil and carfentanil using a common intermediate where the process is potentially safer to the environment when compared to presently known processes.

### BACKGROUND OF THE INVENTION

Analgesics, such as remifentanil and carfentanil, have been prepared in synthetic processes comprising six and seven steps. Examples of such processes are outlined in U.S. Pat. Nos. 5,106,983 and 5,019,583. However, these syntheses often require many steps and unsafe chemical reagents, resulting in increased process costs due to reduced production efficiency, additional material costs, and costs related to the handling of hazardous chemicals.

An alternate process having potentially improved efficiency and the potential for using more environmentally safe materials would be welcome.

### SUMMARY OF THE INVENTION

Among the several features of the present invention, therefore, can be noted the provision of a process for synthesizing intermediates and final synthetic opiate or opioid compounds such as, for example, remifentanil, carfentanil, sufentanil, fentanyl, and alfentanil; the provision of preparing an analgesic or anesthetic; the provision of a process that potentially requires fewer steps for synthesizing remifentanil; the provision of a process that potentially requires fewer steps for synthesizing carfentanil; and the provision of such a process wherein remifentanil is prepared from a substituted piperidine.

Briefly, therefore, the present invention is directed to a process for the preparation of an analgesic or anesthetic. Specifically, the process comprises reacting a compound (I) having the formula:

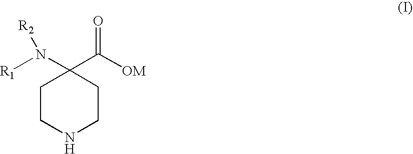

wherein R₁ and R₂ are independently selected from the group consisting of hydrogen, hydrocarbyl and substituted hydrocarbyl and M is hydrogen or a cation, with alcohol, R₃OH, to form intermediate compound (II):

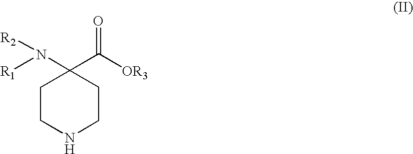

wherein R₃ is hydrocarbyl or substituted hydrocarbyl. The intermediate compound (II) is then reacted with a nitrogen protecting group to form intermediate compound (III):

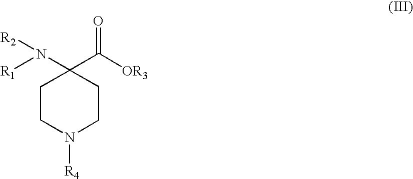

wherein R₄ is hydrocarbyl or substituted hydrocarbyl. The intermediate compound (III) is then acylated to form intermediate compound (IV):

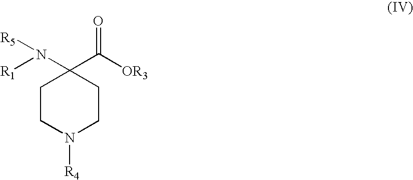

wherein R₅ is —C(O)—R₆ and R₆ is hydrocarbyl or substituted hydrocarbyl. Intermediate compound (IV) is then deprotected to form intermediate compound (V):

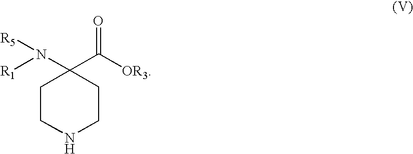

The intermediate compound (V) is then alkylated to form the end product, compound (VI), having the formula:

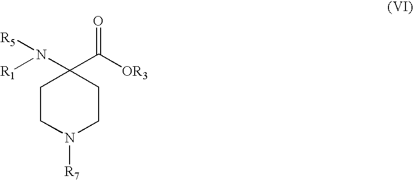

wherein R₇ is hydrocarbyl or substituted hydrocarbyl.

Other aspects and features of this invention will be in part apparent and in part pointed out hereinafter.

### DETAILED DESCRIPTION

In accordance with the present invention, an alternate process for synthesizing analgesics or anesthetics has been discovered. The improved process potentially reduces the process steps required to synthesize the analgesics or anesthetics, improves efficiency and avoids the use of cyanide compounds.

In one embodiment, the process of the present invention results in the synthesis of a compound having the formula (VI):

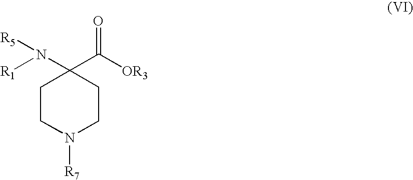

wherein R₅ is —C(O)R₆, R₁ is hydrogen, hydrocarbyl, or substituted hydrocarbyl, and R₃, R₆, and R₇ are independently hydrocarbyl or substituted hydrocarbyl.

In another embodiment, R₇ is hydrocarbyl or substituted hydrocarbyl, R₁ is phenyl or substituted phenyl, R₅ is a carbonyl alkyl, and R₃ is hydrocarbyl or substituted hydrocarbyl.

In one embodiment, the present invention can be used to synthesize remifentanil, chemically identified as 3-[4-methoxycarbonyl-4-[(1-oxopropyl)phenylamino]-1-piperidine]propanoic acid methyl ester, having the formula (VII), utilizing a substituted piperidine starting material.

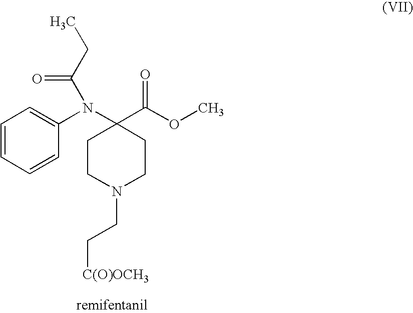

In another embodiment, the present invention can be used to synthesize carfentanil, chemically identified as 4((1-oxopropyl)phenylamino)-1-(2-phenylethyl)-4-piperidinecarboxylic acid, methyl ester, having the formula (VIII), by utilizing a substituted piperidine starting material.

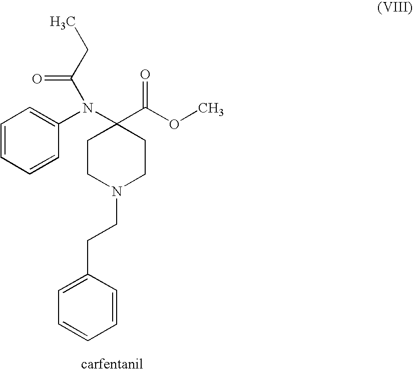

The alternate process of the present invention for synthesizing opiate or opioid analgesics and anesthetics includes the synthesis of a series of intermediates, each of which may be used in the preparation of synthetic opiate or opioid compounds. Scheme 1, below, illustrates a first step in the process wherein a substituted 4-piperidine, compound (I), is reacted with an alcohol to form intermediate compound (II).

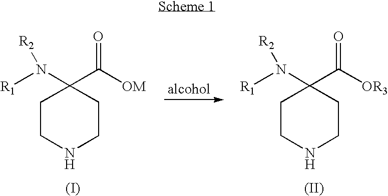

In Scheme 1, compound (I) is reacted with an alcohol, R₃OH, to form intermediate compound (II), wherein R₁ and R₂ are independently selected from the group consisting of hydrogen, hydrocarbyl and substituted hydrocarbyl and R₃ is hydrocarbyl or substituted hydrocarbyl.

In one embodiment, R₁ and R₂ are independently selected from the group consisting of H, aryl, substituted aryl, C₁₋₁₈alkyl, cycloalkyl, substituted cycloalkyl, heterocyclic, R₁₄OR₁₅—, and R₁₆R₁₅—, wherein R₁₄ and R₁₅ are independently hydrocarbyl or substituted hydrocarbyl, and R₁₆ is selected from the group consisting of cycloalkyl, substituted cycloalkyl, and heterocyclic. Preferably, R₁₄ and R₁₅ are independently substituted or unsubstituted alkyl, alkoxy, alkenyl, alkenyloxy, or aryl, R₁₆ is C₃₋₆ cycloalkyl, substituted C₃₋₆ cycloalkyl, or a 5- to 7-membered heterocyclic comprising 1 to 5 heteroatoms selected from oxygen, sulfur, and nitrogen; more preferably, R₁₄ and R₁₅ are independently H, substituted or unsubstituted alkyl, alkoxy, or aryl; still more preferably, R₁ and R₂ are independently selected from H, lower-alkyl, and phenyl.

Typically, R₃ is selected from the group consisting of C₁₋₁₈ hydrocarbyl, R₁₇OR₁₈—, R₁₉R₁₈—, and R₂₀R₁₈—, wherein R₁₇ and R₁₈ are independently hydrocarbyl or substituted hydrocarbyl, R₁₉ is aryl or substituted aryl, and R₂₀ is cycloalkyl, substituted cycloalkyl or heterocyclic. Preferably, R₁₇ and R₁₈ are independently substituted or unsubstituted alkyl, alkenyl, or alkynyl wherein the hydrocarbon chain contains 1 to 18 carbon atoms, R₁₉ is aryl or substituted aryl, R₂₀ is C₃₋₆ cycloalkyl, substituted C₃₋₆ cycloalkyl or a 5- to 7-membered heterocyclic comprising 1 to 5 heteroatoms selected from oxygen, sulfur, and nitrogen; more preferably, R₁₇ and R₁₈ are independently substituted or unsubstituted alkyl. In one preferred embodiment, R₃ is C₁₋₆ alkyl; preferably, methyl, ethyl or propyl.

M corresponds to hydrogen or a cation. Preferably, M is hydrogen or an alkali or alkaline earth metal cation; more preferably, M is hydrogen or a sodium, potassium, or lithium cation; and even more preferably, M is hydrogen.

In one embodiment, the temperature of the reaction mixture during the reaction ranges from about 25° C. to about 80° C., preferably, from about 50° C. to about 70° C. The reaction mixture is permitted to react up to a few days. In one example, the reaction occurs from about 8 to about 100 hours, preferably, from about 24 to about 60 hours.

A desiccant may be used to enhance the rate of esterification of compound (I). Non-limiting examples of desiccants include trimethyl orthoformate, sulfur trioxide, polyphosphoric acid, phosphorous pentoxide, molecular sieves, alumina, silica gel, sodium sulfate anhydrous, magnesium sulfate, and the like.

Independent of whether or not a dessicant is used, a catalyst may be used to enhance the reaction. The catalyst may be selected from the group commonly known as Bronsted acids. A Bronsted acid may be an inorganic acid (e.g., sulfuric acid, hydrochloric acid, nitric acid, phosphoric acid, hydroiodic acid, hydrobromic acid, and hydrofluoric acid) or an organic acid (e.g., methanesulfonic acid, toluenesulfonic acid, benzenesulfonic acid, trifluoroacetic acid, pentafluoroacetic acid, chloroacetic acid, dichloroacetic acid, trichloroacetic acid, and oxalic acid). The catalyst may also be selected from the group known as Lewis acids (e.g., boron trifluoride, aluminum chloride, zinc chloride, tin chloride, titanium tetrachloride and solid acid, such as cationic resins, alumina, silica gel, and others known in the art).

In one embodiment, the reaction mixture comprises about 2 molar equivalents to about 100 molar equivalents of alcohol, optionally about 1 molar equivalent to about 5 molar equivalents of desiccant, and optionally about 1 molar equivalent to about 10 molar equivalents of catalyst per molar equivalent of compound (I).

In another embodiment, the reaction mixture comprises about 4 molar equivalents to about 50 molar equivalents of alcohol, about 1 molar equivalent to about 3 molar equivalents of desiccant, and about 2 molar equivalents to about 4 molar equivalents of catalyst per molar equivalent of compound (I).

Depending on its physical properties, compound (II) may be purified and isolated by extraction, chromatography, distillation, or any combination of methods known in the art. In one embodiment, compound (II) is isolated by the addition of base and water, followed by solvent extraction of compound (II) and finally drying by evaporation.

In another embodiment, compound (II) is isolated by cooling the reaction to below 10° C., adding triethylamine to precipitate the resulting anion of an appropriate Bronsted acid used as the catalyst, filtering the precipitant, and concentrating the residual solution by vacuum. The concentrated solution is then filtered, washed with solvent, and concentrated by vacuum again to obtain compound (II).

Scheme 2, below, illustrates a second step in the process of the present invention wherein intermediate compound (III) is synthesized.

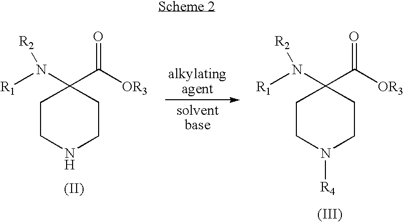

In Scheme 2, compound (II) is mixed with an alkylating agent or a nitrogen protecting agent in the presence of a solvent and a base to form intermediate compound (III), wherein R₄ is hydrocarbyl or substituted hydrocarbyl. Typically, R₄ is selected from the group consisting of aryl, substituted aryl, aralkyl, C₁₋₁₈ alkyl, R₂₁OC(O)R₂₂—, R₂₁C(O)OR₂₂—, R₂₁OR₂₃OC(O)R₂₂—, R₂₄R₂₂—, and R₂₅R₂₂—, wherein R₂₁, R₂₂, and R₂₃ are independently hydrocarbyl or substituted hydrocarbyl, R₂₄ is cycloalkyl or substituted cycloalkyl, and R₂₅ is heterocyclic. Preferably, R₂₁, R₂₂, and R₂₃ are independently alkyl, alkoxy, alkenyl, aryl, aralkyl, or alkenyloxy, R₂₄ is C₅₋₇ cycloalkyl, and R₂₅ is a 5- to 7-membered heterocyclic; more preferably, R₂₁, R₂₂, and R₂₃ are independently linear or branched alkyl, alkoxy, alkenyl, or alkenyloxy having about 1 to about 18 carbon atoms or an aryl or aralkyl, R₂₄ is C₅₋₇ cycloalkyl, and R₂₅ is a 5- to 7-membered heterocyclic comprising 1 to 5 heteroatoms selected from oxygen, sulfur, and nitrogen. More preferably, R₄ is benzyl, substituted benzyl, phenyl, substituted phenyl (e.g., 2-phenylethyl), methyl propionyl, ethyl propionyl, 2-(2-thienyl)ethyl, or 2-(4-ethyl-4,5-dihydro-5-oxo-1H-tetrazol-1-yl)ethyl.

General examples of alkylating agents include compounds having the structure:

L-R₂₆—R₂₇

wherein L is a displacement or leaving group. In one embodiment, L, R₂₆, and R₂₇ are independently hydrocarbyl or substituted hydrocarbyl. Typically, L is a halide, toluenesulfonate, or methylsulfonate; R₂₆ is hydrocarbyl or substituted hydrocarbyl having 1 to 18 carbons; and R₂₇ is selected from R₂₁OC(O)R₂₂—, R₂₁C(O)OR₂₂—, R₂₁OR₂₃OC(O)R₂₂—, R₂₄R₂₂—, and R₂₅R₂₂—, wherein R₂₁, R₂₂, R₂₃, R₂₄, and R₂₅, are as defined above. Preferably, R₂₆ is methyl or ethyl, and R₂₇ is —C(O)OCH₃, —C(O)OCH₂CH₃, phenyl, -2-(2-thienyl), or -2-(4-ethyl-4,5-dihydro-5-oxo-1H-tetrazol-1-yl)ethyl.

The alkylating agents may also comprise an electron deficient moiety to an electron withdrawing group such as carbonyl, nitrile, carbonyloxy, alkyl carbonate, and alkyl-alkoxy carbonate. Non-limiting specific examples of alkylating agents include methyl acrylate, ethyl acrylate, acrylic acid, acryronitrile, acrylamide, acrolein, phenylethyl halide, tolylate, mesylate, styrene, and substituted styrene. Alkylating agents comprising an electron deficient moiety may be depicted as follows:

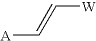

wherein A is hydrogen, hydrocarbyl, or substituted hydrocarbyl and W is hydrocarbyl, substituted hydrocarbyl, nitrile, or amide. In one example, A is hydrogen, linear or branched C₁₋₁₈ alkyl, aryl, substituted aryl, alkylaryl, C₅₋₇ cycloalkyl or substituted C₅₋₇ cycloalkyl; and W is carboxylic acid, carboxylic acid ester, nitrile, amide, carbonyl, or aryl. Most preferably A is hydrogen and W is a carboxylic acid ester or aryl.

Examples of the base used in the reaction of Scheme 2 include metal hydroxide, metal alkoxide, metal hydride, metal carbonate, metal hydrogen carbonate, amine, quaternary alkyl ammonia hydroxide, and ammonia. Examples of metal alkoxides and metal hydrides include sodium, potassium, cesium, magnesium, aluminum alkoxides and hydrides and the like. Preferably, the base is quaternary alkylammonium hydroxide, trialkylamine, or a metal alkoxide.

The solvent of Scheme 2 is an organic solvent. Typical solvents include, dimethyl sulfoxide, ether, dichloromethane, chloroform, carbon tetrachloride, ethylene chloride, acetonitrile, toluene, ethylacetate, propylacetate, butylacetate, alcohol ethers, HMPA (hexamethyl phosphoramide), HMPT (hexamethyl phosphorimidic triamide), alkanols containing 1 to 18 carbon atoms, C₁₋₁₈ hydrocarbyl, aryl-alcohol, and 5- to 7-membered heterocyclic alcohols comprising 1 to 5 heteroatoms selected from oxygen, sulfur, and nitrogen. Most preferable solvents are selected from the group consisting of acetonitrile, chloroform, 1,2-dichloroethane, 1,1,2-trichloroethane, dichloromethane, and carbon tetrachloride.

In one embodiment, the reaction mixture comprises about 1 molar equivalent to about 5 molar equivalents of alkylating agent and about 1 molar equivalent to about 5 molar equivalents of base per molar equivalent of compound (II). Preferably, the reaction mixture comprises about 1 to about 3 equivalents of an alkylating agent and about 1 equivalent to about 3 equivalents of base per molar equivalent of compound (II).

The solvent to compound (II) ratio on a volume to weight basis is about 1:2 to about 1:100; preferably, the solvent to compound (II) ratio is about 1:4 to about 1:50.

In one embodiment, the temperature of the reaction mixture during the reaction ranges from about −10° C. to about 65° C. In another embodiment, the reaction temperature ranges from about 10° C. to about 40° C. The reaction mixture is permitted to react up to a couple of days. In one example, the reaction is carried out up to about 24 hours. In another example, the reaction time is less than about 12 hours. In still another example, the reaction time is from about 2 hours to about 6 hours.

In one embodiment, methyl acrylate was added to compound (II) dispersed in methanol. Triethylamine was added and mixed for 1 hour. The resulting solid was filtered off and the methanolic solution concentrated by vacuum to obtain compound (III). Compound (III) may be further purified through recrystallization with organic solvents, preparative chromatography or a combination of methods.

Scheme 3, below, illustrates a third step in the process of the present invention wherein intermediate compound (IV) is synthesized.

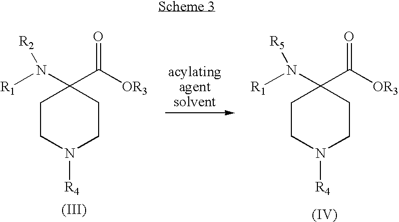

In Scheme 3, compound (III) is reacted with an acylating agent in a reaction mixture containing a solvent to form compound (IV), wherein R₅ is an acyl moiety corresponding to the acylating agent.

The temperature of the reaction mixture ranges from about 20° C. to about 80° C. In another example, the reaction temperature ranges from about 40° C. to about 65° C. The reaction mixture is permitted to react from about 4 hours to about 18 hours. In one example, the reaction is carried out from about 4 hours to about 8 hours.

In one embodiment, R₅ is —CO—R₆ and R₆ is hydrocarbyl or substituted hydrocarbyl. In one example of this embodiment, the acylating agent is an acid halide, preferably a C₁₋₁₈ acid halide selected from alkyl acid halides and alkoxy-alkyl halides. Examples of acylating agents include, but are not limited to, acetyl chloride, acetic anhydride, propionyl chloride, propionic anhydride, methyl ketene, butanoyl chloride, alkyl acid cyanides, and the like. In one embodiment, the alkyl group comprises between 1 and about 18 carbon atoms. In another embodiment, the alkyl group comprises less than about 6 carbon atoms. In yet another embodiment, the alkyl group comprises between 2 and 4 carbon atoms. Preferably the acylating agent is propionyl chloride or propionic anhydride.

The solvent contained in the reaction mixture can be any solvent that is inert to the reaction occurring in Scheme 3. Examples of such solvents include, but are not limited to, acetonitrile; acetone; dichloromethane; chloroform; n,n-dimethylformamide; dimethylsulfoxide; ethylacetate; dichloroethane; aromatic hydrocarbons (e.g., benzene, toluene, and xylene), lower alkanols (e.g., methanol, ethanol, isopropanol, n-propanol, 1-butanol, tert-butanol); ketones (e.g., 4-methyl-2-pentanone); ethers (e.g., 1,4-dioxane, tetrahydrofuran (THF), 1,1-oxybisethane), nitrobenzene; and mixtures thereof. In one example, the reaction mixture comprises acetonitrile, dichloromethane, lower alkanols, or mixtures thereof. In another example, the reaction mixture comprises acetonitrile.

The reaction mixture optionally contains an acid scavenger. The acid scavenger may include metal hydrides, hydroxides, carbonates, bicarbonates, amines, and the like.

In one embodiment, the reaction mixture comprises about 1 molar equivalent to about 50 molar equivalents of acylating agent per molar equivalent of compound (III). Preferably, the reaction mixture comprises about 2 to about 5 molar equivalents of an acylating agent per molar equivalent of compound (III). The solvent to compound (III) ratio on a volume to weight basis is about 1:4 to about 1:50, preferably, the solvent to compound ratio is about 1:4 to about 1:25.

Compound (IV) is collected by filtration and drying. The product may be purified by methods known in the art including recrystallization and/or solvent extraction.

Scheme 4, below, illustrates a fourth step in the process of the present invention wherein intermediate compound (V) is synthesized.

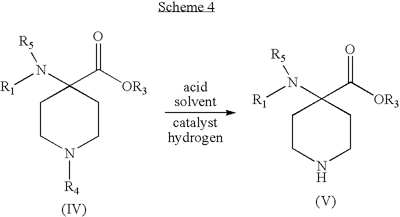

In scheme 4, the nitrogen protecting group is removed. For example, when R₄ is benzyl, compound (IV), with or without a solvent, may be reacted with an acid and a catalyst in a hydrogenator (pressure reactor under hydrogen) to remove R₄.

The temperature of the reaction mixture ranges from about 25° C. to about 120° C. In another example, the reaction temperature ranges from about 50° C. to about 100° C. The reaction mixture is permitted to react from about 8 hours to about 100 hours. In one example, the reaction is carried out from about 8 hours to about 48 hours. In another example, the reaction is carried out in about 24 hours or less.

In one embodiment, the acid is acetic acid, propionic acid, or phosphoric acid.

The catalyst is typically a heterogeneous transition metal catalyst such as platinum, palladium, rhodium, etc. The transition metal may be in a supported form (e.g., on carbon, alumina, silica, etc.).

When present, the solvent of Scheme 4 is an organic solvent or water. Preferred solvents include water, alcohols and organic acids. In one embodiment, the solvent is acetic acid.

Typically, the reaction mixture comprises 0 molar equivalents to about 100 molar equivalents of solvent and about 1 molar equivalent to about 100 molar equivalents of acid per molar equivalent of compound (IV). Preferably, the reaction mixture comprises about 1 to about 20 equivalents of solvent and about 1 equivalent to about 20 equivalents of acid per molar equivalent of compound (IV).

Scheme 5, below, illustrates a fifth step in the process of the present invention wherein the final compound, compound (VI), is synthesized.

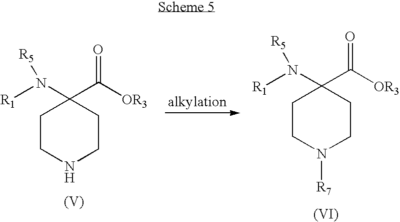

In Scheme 5, compound (V) is alkylated to form compound (VI). This alkylation step may be carried out via conventional methods known in the art. In one embodiment, compound (V) is converted to compound (VI) via any one of the methods described in U.S. Pat. No. 5,019,583 (see, col. 4, line 33-col. 15, line 47 of U.S. Pat. No. 5,019,583, which is hereby incorporated by this reference). For example, an appropriate R₇ group may be introduced to compound (V) by the alkylation reaction of compound (V) with an appropriate halide.

Typically, compound (V) is mixed in with an alkylating agent in the presence of a solvent and a base to form compound (VI), wherein R₇ is hydrocarbyl or substituted hydrocarbyl. Preferably, R₇ is selected from the group consisting of aryl, aralkyl, C₁₋₁₈ alkyl, R₂₈OC(O)R₂₉—, R₂₈C(O)OR₂₉—, R₂₈OR₃₀OC(O)R₂₉—, R₃₁R₂₉—, and R₃₂R₂₉—, wherein R₂₈, R₂₉, and R₃₀ are independently hydrocarbyl or substituted hydrocarbyl, R₃₁ is cycloalkyl, substituted cycloalkyl, aryl or substituted aryl, and R₃₂ is heterocyclic. Typically, R₂₈, R₂₉, and R₃₀ are independently alkyl, alkoxy, alkenyl, or alkenyloxy, R₃₁ is C₅₋₇ cycloalkyl, phenyl or substituted phenyl and R₃₂ is a 5- to 7-membered heterocyclic; more preferably, R₂₈, R₂₉, and R₃₀ are independently linear or branched alkyl, alkoxy, alkenyl, or alkenyloxy having 1 to about 18 carbon atoms, R₃₁ is C₅₋₇cycloalkyl, phenyl or substituted phenyl, and R₃₂ is a 5- to 7-membered heterocyclic comprising 1 to 5 heteroatoms selected from oxygen, sulfur, and nitrogen. In one preferred embodiment, R₇ is methyl propionyl, ethyl propionyl, 2-phenylethyl, 2-(2-thienyl)ethyl, or 2-(4-ethyl-4,5-dihydro-5-oxo-1H-tetrazol-1-yl)ethyl.

A general description of the possible alkylating agents is discussed above in detail for Scheme 2.

Examples of the base typically used in the reaction of Scheme 5 include metal hydroxide, metal alkoxide, metal hydride, metal carbonate, metal hydrogen carbonate, amine, quaternary alkyl ammonia hydroxide, and ammonia. Examples of metal alkoxides and metal hydrides include sodium, potassium, cesium, magnesium, aluminum alkoxides and hydrides and the like. Preferably, the base is ammonia or a metal alkoxide.

The solvent of Scheme 5 is typically an organic solvent. Preferred solvents include, dimethyl sulfoxide, ether, dichloromethane, chloroform, carbon tetrachloride, ethylene chloride, acetonitrile, toluene, ethylacetate, propylacetate, butylacetate, alcohol ethers, alkanols containing 1 to 18 carbon atoms, hydrocarbons containing 1 to 18 carbon atoms, aryl-alcohol, and 5- to 7-membered heterocyclic alcohols comprising 1 to 5 heteroatoms selected from oxygen, sulfur, and nitrogen. Most preferable solvents include acetonitrile, chloroform, 1,2-dichloroethane, 1,1,2-trichloroethane, dichloromethane, and carbon tetrachloride.

In one embodiment, the reaction mixture comprises about 1 molar equivalent to about 5 molar equivalents of alkylating agent and about 1 molar equivalent to about 5 molar equivalents of base per molar equivalent of compound (V). Preferably, the reaction mixture comprises about 1 to about 3 equivalents of an alkylating agent and about 1 equivalent to about 3 equivalents of base per molar equivalent of compound (V).

The solvent to compound (V) ratio on a volume to weight basis is about 1:2 to about 1:100, preferably, the solvent to compound ratio is about 1:4 to about 1:50.

In one embodiment, the temperature of the reaction mixture during the reaction ranges from about −10° C. to about 65° C. In another embodiment, the reaction temperature ranges from about 10° C. to about 40° C. The reaction mixture is permitted to react up to a couple of days. In one example, the reaction is carried out up to about 24 hours. In another example, the reaction is carried out in less than about 12 hours. In still another example, the reaction time is from about 2 hours to about 6 hours.

In one embodiment, methyl acrylate was added to compound (V) dispersed in methanol. Triethylamine was added and mixed for 1 hour. The resulting solid was filtered off and the methanolic solution concentrated by vacuum to obtain compound (VI). Compound (VI) may be further purified through recrystallization with organic solvents, preparative chromatography or a combination of methods.

The overall process of the present invention for synthesizing opiate or opioid analgesics and anesthetics that incorporates the individual steps described above is illustrated in Scheme 6, below.

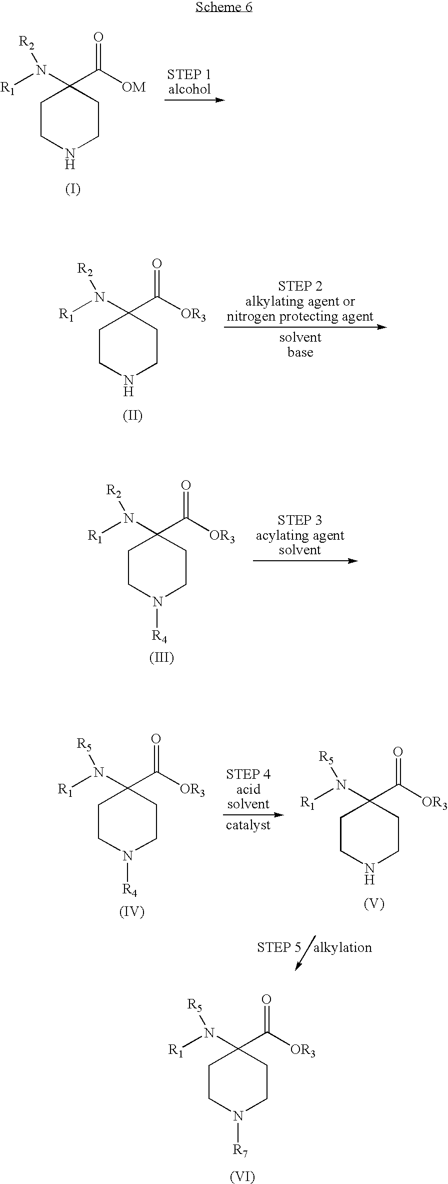

In one embodiment of the present invention, a process for synthesizing remifentanil is provided. An illustration of this process is shown below in Scheme 7.

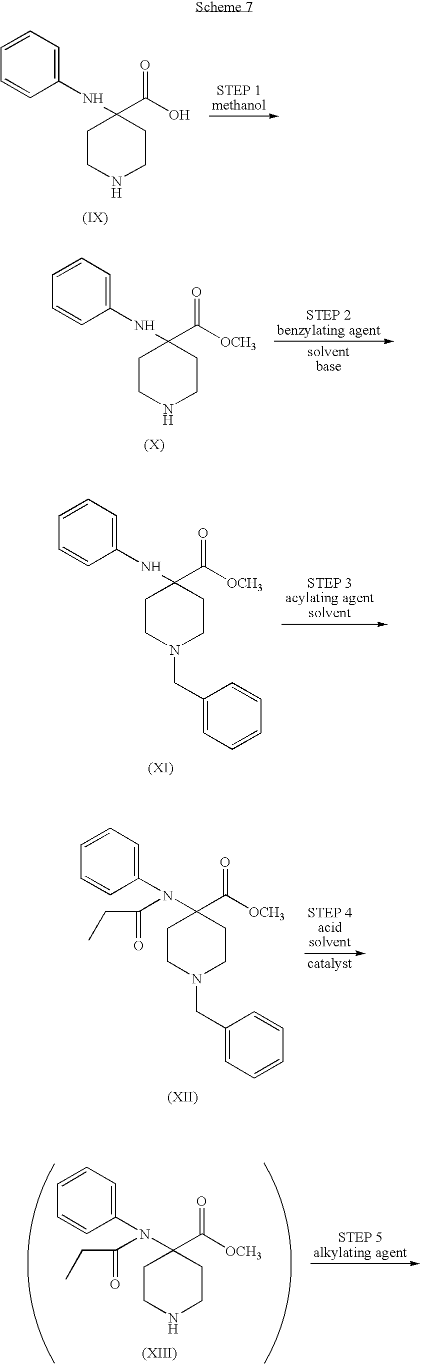

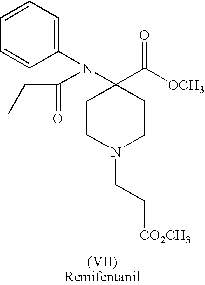

In Step 1, compound (IX), for example, N-phenyl-α-(4-piperidino)glycine, is reacted in a reaction mixture with methanol to form compound (X). The reaction may optionally be carried out in the presence of a catalyst and/or desiccant.

In one embodiment, the reaction mixture comprises about 2 molar equivalents to about 100 molar equivalents of methanol per molar equivalent of compound (IX). In another embodiment, the reaction mixture comprises about 4 molar equivalents to about 50 molar equivalents of methanol per molar equivalent of compound (IX).

The temperature of the reaction mixture during the reaction ranges from about 25° C. to about 80° C. In another example, the reaction temperature ranges from about 50° C. to about 70° C. The reaction mixture is permitted to react up to a few days. In one example, the reaction is from about 8 to about 100 hours. Preferably, the reaction time is from about 24 hours to about 60 hours.

Desiccant can be used to enhance the rate of esterification of compound (IX). Non-limiting examples of desiccants include trimethyl orthoformate, sulfur trioxide, polyphosphoric acid, phosphorous pentoxide, molecular sieves, alumina, silica gel, sodium sulfate anhydrous, magnesium sulfate, and the like. In one embodiment, the desiccant is trimethyl orthoformate. In one example the reaction mixture is charged with about 1 molar equivalent to about 5 molar equivalents of desiccant, per molar equivalent of compound (IX), preferably 1 molar equivalent to about 3 molar equivalents of desiccant per molar equivalent of compound (IX).

The catalyst can be selected from the group commonly known as Bronsted acids or Lewis acids. In one embodiment the catalyst is sulfuric acid. In one embodiment the reaction mixture comprises about 1 molar equivalent to about 10 molar equivalents of the catalyst per molar equivalent of compound (IX).

In one embodiment, compound (X) is isolated by neutralizing the reaction with solid sodium carbonate and water, followed by solvent extraction with ethyl acetate, which is then separated, air dried, and re-dissolved in methanol to yield purified compound (X) in the methanol solution. In an alternative embodiment, compound (X) is isolated by cooling the reaction to below 10° C., adding triethylamine to precipitate the resulting anion of an appropriate Bronsted acid used as the catalyst, filtering the precipitant, and concentrating the residual solution by vacuum. The concentrated solution is then filtered, washed with solvent, and concentrated by vacuum again to obtain compound (X).

In step 2, compound (X), for example, N-phenyl-α-(4-piperidino)glycine methyl ester, is mixed with benzyl halide, benzyl alkyl sulfonate, or benzyl aryl sulfonate, in the presence of a solvent and a base to form compound (XI).

In one embodiment, the reaction mixture comprises about 1 molar equivalent to about 5 molar equivalents of benzylating agent and about 1 molar equivalent to about 5 molar equivalents of base per molar equivalent of compound (X). Preferably, the reaction mixture comprises about 1 to about 3 molar equivalents of benzylating agent and about 1 to about 3 molar equivalents of base per molar equivalent of compound (X). The solvent to compound (X) ratio on a weight to volume basis is about 1:2 to 1:100, preferably, the solvent to compound ratio is 1:4 to 1:50.

The temperature of the reaction mixture during the reaction ranges from about −10° C. to about 65° C. In another embodiment, the reaction temperature ranges from about 10° C. to about 40° C. The reaction mixture is permitted to react up to a couple of days. In one example, the reaction is carried out about 24 hours. In another example, the reaction time is from about 2 hours to about 6 hours.

Preferred solvents are selected from the group consisting of acetonitrile, chloroform, 1,2-dichloroethane, 1,1,2-trichloroethane, dichloromethane and carbon tetrachloride.

In one embodiment, benzyl chloride is added to compound (X) dispersed in acetonitrile, and triethylamine is added and mixed for 1 hour. The resulting solid is filtered off and the acetonitrile solution is concentrated by vacuum to obtain compound (XI). Compound (XI) may be further purified through recrystallization with organic solvents, preparative chromatography, or a combination of methods.

In Step 3, compound (XI) is reacted with an acylating agent in a reaction mixture containing a solvent to form compound (XII). Preferably the acylating agent is propionyl chloride or propionic anhydride.

The temperature of the reaction mixture ranges from about 20° C. to about 80° C. In another example, the reaction temperature ranges from about 40° C. to about 65° C. The reaction mixture is permitted to react from about 4 hours to about 18 hours, preferably from about 4 hours to about 8 hours.

The solvent contained in the reaction mixture can be any solvent that is inert to the reaction occurring in Step 3. Examples of such solvents include, but are not limited to acetonitrile; acetone; dichloromethane; chloroform; n,n-dimethylformamide; dimethylsulfoxide; ethylacetate; dichloroethane; aromatic hydrocarbons (e.g., benzene, toluene, and xylene), ketones (e.g., 4-methyl-2-pentanone), ethers (e.g., 1,4-dioxane, tetrahydrofuran (THF), 1,1-oxybisethane), nitrobenzene; and mixtures thereof. In one example, the reaction mixture comprises acetonitrile.

In one embodiment, the reaction mixture comprises about 1 molar equivalent to about 50 molar equivalents of acylating agent per molar equivalent of compound (XI). Preferably, the reaction mixture comprises about 2 to about 5 molar equivalents of an acylating agent per molar equivalent of compound (XI). The solvent to compound (XI) ratio on a volume to weight basis is about 1:4 to about 1:25, preferably, the solvent to compound ratio is 1:4 to 1:15.

In Step 4, compound (XII) is reacted with hydrogen, in a reaction mixture containing an acid and catalyst and optionally a solvent to form compound (XIII).

The temperature of the reaction mixture ranges from about 25° C. to about 120° C. In another example, the reaction temperature ranges from about 50° C. to about 100° C. The reaction mixture is permitted to react from about 8 hours to about 100 hours. In one example, the reaction is carried out from about 8 hours to about 48 hours.

Preferably, the acid is acetic acid, propionic acid, or phosphoric acid.

The catalyst is typically a heterogeneous transition metal catalyst. Preferably, the catalyst is selected from the group consisting of platinum, palladium, and rhodium.

Preferably, Step 4 is conducted in water or an organic solvent. Common organic solvents include dimethyl sulfoxide, ether, dichloromethane, chloroform, carbon tetrachloride, ethylene chloride, acetonitrile, toluene, ethylacetate, propylacetate, butylacetate, alcohol ethers, alkanols containing 1 to 18 carbon atoms, hydrocarbons containing 1 to 18 carbon atoms, aryl-alcohol, and 5- to 7-membered heterocyclic alcohols comprising 1 to 5 heteroatoms selected from oxygen, sulfur, and nitrogen.

In another embodiment, Scheme 7 is modified to prepare carfentanil. The method of preparing carfentanil is nearly identical to that of remifentanil with the exception of step 5 wherein the alkylating compound used to produce carfentanil is styrene or phenylethyl halide.

### EXAMPLES

The following examples are provided in order to more fully illustrate the present invention.

### Examples of Step 1

(A) 30 ml of concentrated sulfuric acid was added slowly to a solution containing 20 g of compound (IX) and 350 ml of methanol below 65° C. The solution was stirred at 65° C. for 2 to 4 days depending on the time allowed. Typically 70% yield occurs in 2 days and 90% yield occurs in 4 days. The solution was cooled to room temperature, then neutralized with concentrated ammonium hydroxide in an ice bath (below 20° C.). The solid was filtered off and the solution was concentrated in vacuum. 50 ml of methanol was added to dissolve the oil followed by 1 g of Darco (activated charcoal) and 10 g of silica gel. The solid was filtered off and the solution was concentrated in vacuum to obtain 11 g of compound (X) as amber oil. The oil solidified upon standing.

(B) 5 g of compound (IX), 9 ml of concentrated hydrochloric acid, and 100 ml of methanol were stirred at reflux (65° C.). The reaction was monitored by liquid chromatography and showed 20% conversion to compound (X) in 25 hours and 40% conversion to compound (X) in 48 hours.

### Example of Step 2

10 g of compound (X), 4.9 ml of benzyl chloride, 6 ml of triethylamine, and 70 ml of acetonitrile were stirred at room temperature overnight. About 100 ml of water and 100 ml of ethylacetate were added to quench the reaction. The layers were separated and collected. The ethylacetate solution was concentrated under vacuum to obtain 5 g of yellow oil. The yellow oil was filtered through a funnel of silica (about 50 g) and eluted with ethylacetate and concentrated in vacuum to obtain 4 g of compound (XI) as yellow oil.

### Example of Step 3

5 g of compound (XI) and 10 ml of propionic anhydride were stirred at 100° C. overnight, then cooled to room temperature. 25 ml of methanol was added and stirred for 4 hours to destroy the excess propionic anhydride then concentrated in vacuum to obtain brown oil. The brown oil was filtered through a funnel of silica gel (about 75 g) and eluted with ethyl acetate (about 500 ml). The ethyl acetate solution was concentrated in vacuum to obtain 2 g of compound (XII) as brown oil.

### Example of Step 4

A crude reaction solution containing 500 mg of compound (XII), 2 ml of methyl propionate, and 60 ml of methanol was transferred to a Parr reactor (450 ml, Hastelloy C). 1 ml of acetic acid and 100 mg of 5% Palladium on carbon were charged. The reaction was hydrogenated at 50° C. overnight. About 50% de-benzylated product (XIII) was observed by LC-MS (M+H=290). The catalyst can be filtered off.

### Examples of Step 5

(A) The methanol solution from step 4 was cooled below 10° C. and methyl acrylate (6 g) was added slowly to maintain the temperature below 40° C. The solution was stirred for 30 minutes, followed by cooling to room temperature. Triethylamine (20 mL) was added and stirred for 1 hour. The resulting solid was filtered off and the methanolic solution was concentrated under vacuum to produce remifentanil.

To prepare carfentanil, the methyl acrylate may be replaced with phenylalkene (e.g, styrene), phenylethyl halide or phenylethyl sulfonate.

(B) Methyl acrylate (20 mL) was added to the suspension which was allowed to warm to room temperature, then stirred at 40° C. for 1 hour, and cooled to room temperature. The resulting solid was filtered off and washed with methanol (100 mL). The remaining solution was concentrated, water (500 mL) added, and the product extracted with dichloromethane (100 mL). The aqueous phase was washed with dichloromethane (50 mL). The dichloromethane solutions were combined and dried over magnesium sulfate and concentrated under vacuum to obtain 40.4 g of pink solid. The pink solid was re-dissolved into 250 mL dichloromethane and filtered through a funnel containing silica gel (70 g) the product eluted with 2 liters of ethyl acetate. The ethyl acetate solution was evaporated under vacuum to dryness to obtain remifentanil

To prepare carfentanil, the methyl acrylate may be replaced with phenylalkene (e.g, styrene), phenylethyl halide or phenylethyl sulfonate.

(C) 1 g of methyl 3-(4-anilino-4-carbomethoxy-piperidino) propionate and 1 ml of propionyl chloride were dissolved in 25 ml of chloroform in a 3-neck round bottom flask equipped with a condenser. The solution was stirred at 65° C. overnight, then cooled to room temperature. Hexane was added until precipitation occurred. The solution was filtered. The product was isolated by suction filtration through a medium frit glass Buchner funnel to obtain 0.7 g of remifentanil hydrochloride.

(D) Methyl acrylate was added to the solution in step 4 with stirring and concentrated ammonium hydroxide is added to neutralize the acid resulting in remifentanil free base in situ. The solid is filtered off and the solution is concentrated in vacuum to obtain crude product. This crude product can be purified by traditional means (e.g., chromatography or re-crystallization) followed by acidification with hydrogen chloride or hydrochloric acid to obtain remifentanil hydrochloride.

### Abbreviations and Definitions

The term “acyl” denotes a radical provided by the residue after removal of hydroxyl from an organic acid, for example, COOH of an organic carboxylic acid, e.g., RC(O)—, wherein R is R₂₄, R₂₄O—, R₂₄R₂₅N—, or R₂₅S—, R₂₄ is hydrocarbyl, heterosubstituted hydrocarbyl, or heterocyclo and R₂₅ is hydrogen, hydrocarbyl or substituted hydrocarbyl. Examples of such acyl radicals include alkanoyl and aroyl radicals. Examples of lower alkanoyl radicals include formyl, acetyl, propionyl, butyryl, isobutyryl, valeryl, isovaleryl, pivaloyl, hexanoyl, and trifluoroacetyl.

The term “alkenyl” denotes a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl. The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” denote linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” denotes an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “amino” as used herein alone or as part of another group denotes the moiety —NR₃₃R₃₄ wherein R₃₃ and R₃₄ are hydrocarbyl, substituted hydrocarbyl or heterocyclo.

The terms “halide,” “halogen,” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties comprise 1 to 18 carbon atoms. They may be straight or branched chain or cyclic and include methyl, ethyl, propyl, isopropyl, allyl, benzyl, hexyl and the like.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, keto, acyl, acyloxy, nitro, tertiaryamino, amido, nitro, cyano, ketals, acetals, esters and ethers.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained.

As various changes could be made in the above methods and products without departing from the scope of the invention, it is intended that all matter contained in the above description and shown in any accompanying drawings shall be interpreted as illustrative and not in a limiting sense.

1-24. (canceled)

25. A process for the preparation of an opiate or opioid analgesic or anesthetic, the process comprising:
reacting a compound (I) having the formula: 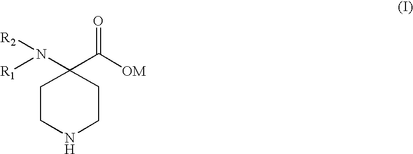
with an alcohol, R₃OH, to form intermediate compound (II) having the formula: 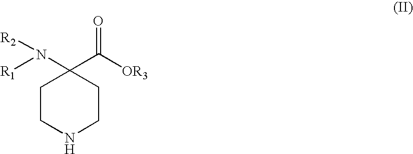
wherein R₁ and R₂ are independently selected from the group consisting of hydrogen, hydrocarbyl and substituted hydrocarbyl, R₃ is hydrocarbyl or substituted hydrocarbyl, and M is hydrogen or a cation;
reacting intermediate compound (H) with a nitrogen protecting group to form intermediate compound (III) having the formula: 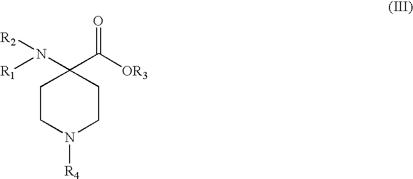
wherein R₄ is hydrocarbyl or substituted hydrocarbyl;
reacting intermediate compound (III) with an acylating agent to form intermediate compound (IV) having the formula: 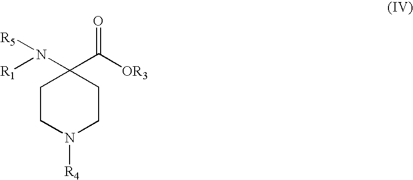
wherein R₅ is —C(O)—R₆ and R₆ is hydrocarbyl or substituted hydrocarbyl;
removing the nitrogen protecting group from intermediate compound (IV) to form intermediate compound (V) having the formula: 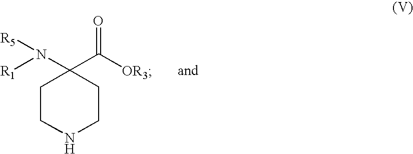
alkylating intermediate compound (V) to form the opioid or opiate compound (VI) having the formula: 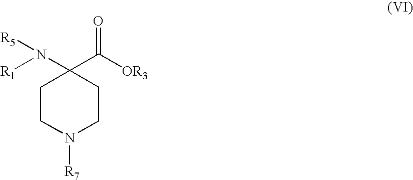
wherein R₇ is hydrocarbyl or substituted hydrocarbyl.

26. The process of claim 25, wherein
R₁ and R₂ are independently H, aryl, substituted aryl, C₁₋₁₈ alkyl, cycloalkyl, substituted cycloalkyl, heterocyclic, R₁₄OR₁₅— or R₁₅R₁₅—;
R₁₄ and R₁₅ are independently hydrocarbyl or substituted hydrocarbyl; and
R₁₆ is cycloalkyl, substituted cycloalkyl, or heterocyclic.

27. The process of claim 26 wherein R₁ is phenyl and R₂ is H.

28. The process of claim 27 wherein M is hydrogen or a metal cation selected from the group consisting of sodium, potassium, and lithium.

29. The process of claim 28 further comprising reacting intermediate compound (I) with an acid, before reacting with an alcohol having the formula R₃OH, to form the intermediate compound (II).

30. The process of claim 29, wherein reacting intermediate compound (III) with an acylating agent to form intermediate compound (IV) occurs in a solvent selected from the group consisting of water, acetonitrile, acetone, dichloromethane, HMPA, HMPT, chloroform, n,n-dimethylformamide, dimethylsulfoxide, ethylacetate, dichloroethane, triethylamine, benzene, toluene, xylene, methanol, ethanol, isopropanol, n-propanol, 1-butanol, tert-butanol, 4-methyl-isopropanol, 1,4-dioxane, tetrahydrofuran (THF), 1,1-oxybisethane, nitrobenzene, and mixtures thereof.

31. The process of claim 30, wherein said solvent is selected from the group consisting of methanol, ethanol, n-propanol, isopropanol, and acetonitrile.

32. The process of claim 31 wherein R₃ is C₁₋₁₈ hydrocarbyl, R₁₇OR₁₈—, R₁₉R₁₈—, or R₂₀R₁₈—;
R₁₇ and R₁₈ are independently hydrocarbyl or substituted hydrocarbyl;
R₁₉ is aryl or substituted aryl; and
R₂₀ is cycloalkyl, substituted cycloalkyl or heterocyclic.

33. The process of claim 32 wherein R₃ is alkyl.

34. The process of claim 33, wherein R₃ is methyl or ethyl.

35. The process of claim 34 wherein R₄ is selected from the group consisting of aryl, substituted aryl, aralkyl, C₁₋₁₈ alkyl, R₂₁OC(O)R₂₂—, R₂₁C(O)OR₂₂—, R₂₁OR₂₃OC(O)R₂₂—, R₂₄R₂₂—, and R₂₅R₂₂—;
R₂₁, R₂₂, and R₂₃ are independently hydrocarbyl or substituted hydrocarbyl;
R₂₄ is cycloalkyl or substituted cycloalkyl; and
R₂₅ is heterocyclic.

36. The process of claim 35 wherein R₂₁, R₂₂, and R₂₃ are independently alkyl, alkoxy, alkenyl, aryl, aralkyl or alkenyloxy;
R₂₄ is C₅₋₇ cycloalkyl; and
R₂₅ is a 5- to 7-membered heterocyclic.

37. The process of claim 36 wherein R₂₁, R₂₂, and R₂₃ are independently linear or branched C₁₋₁₈ alkyl, C₁₋₁₈ alkoxy, C₂₋₁₈ alkenyl, or C₂₋₁₈ alkenyloxy;
R₂₄ is C₅₋₇ cycloalkyl; and
R₂₅ is a 5- to 7-membered heterocyclic comprising 1 to 5 heteroatoms selected from oxygen, sulfur, and nitrogen.

38. The process of claim 37 wherein R₄ is benzyl, substituted benzyl, phenyl, substituted phenyl, alkyl propionyl, 2-(2-thienyl)alkyl, or 2-(4-ethyl-4,5-dihydro-5-oxo-1H-tetrazol-1-yl)alkyl.

39. The process of claim 38 wherein R₇ is selected from the group consisting of aryl, aralkyl, C₁₋₁₈ alkyl, R₂₈OC(O)R₂₉—, R₂₈C(O)OR₂₉—, R₂₈OR₃₀OC(O)R₂₉—, R₃₁R₂₉—, and R₃₂R₂₉—;
R₂₈, R₂₉, and R₃₀ are independently hydrocarbyl or substituted hydrocarbyl;
R₃₁ is cycloalkyl or substituted cycloalkyl; and
R₃₂ is heterocyclic.

40. The process of claim 39 wherein R₇ is selected from the group consisting of methyl propionyl, ethyl propionyl, 2-phenylethyl, 2-(2-thienyl)ethyl, and 2-(4-ethyl-4,5-dihydro-5-oxo-1H-tetrazol-1-yl)ethyl.

41. The process of claim 40 wherein compound (II) is formed in the presence of a catalyst.

42. The process of claim 41, wherein the alkylating agent is selected from the group consisting of methyl acrylate, ethyl acrylate, acrylic acid, acryronitrile, acrylamide, acrolein, phenylethyl halide, tolylate, mesylate, styrene, and substituted styrene.

43. The process of claim 42 wherein the acylating agent is selected from the group consisting of acetyl chloride, acetic anhydride, ethanoyl chloride, propionyl chloride, propionic anhydride, methyl ketene, butanoyl chloride, and an alkyl acid cyanide.

44. The process of claim 43 wherein compound (VI) is remifentanil or carfentanil.
